SEQUENCE LISTING (24 sequences)

SEQ ID NO: 1 - RNA, 100 nt, synthetic construct
  misc_feature 1..100: note = SARS-CoV-2 N1 (1-3, 97-99: 2'-O-methyl/ phosphorothioate modification)
  source 1..100: mol_type = other RNA
aaacgtaatgcggggtgcatgttttagagctagaaatagcaagttaaaataaggctagtccgttatcaacttgaaaaagtggcaccgagtcggtgctttt

SEQ ID NO: 2 - RNA, 100 nt, synthetic construct
  misc_feature 1..100: note = SARS-CoV-2 N2 (1-3, 97-99: 2'-O-methyl/ phosphorothioate modification)
  source 1..100: mol_type = other RNA
tgggggcaaattgtgcaattgttttagagctagaaatagcaagttaaaataaggctagtccgttatcaacttgaaaaagtggcaccgagtcggtgctttt

SEQ ID NO: 3 - RNA, 100 nt, synthetic construct
  misc_feature 1..100: note = SARS-CoV-2 N3 (1-3, 97-99: 2'-O-methyl/ phosphorothioate modification)
  source 1..100: mol_type = other RNA
gggtgccaatgtgatcttttgttttagagctagaaatagcaagttaaaataaggctagtccgttatcaacttgaaaaagtggcaccgagtcggtgctttt

SEQ ID NO: 4 - RNA, 100 nt, synthetic construct
  misc_feature 1..100: note = pH1N1 H1 (1-3, 97-99: 2'-O-methyl/ phosphorothioate modification)
  source 1..100: mol_type = other RNA
ccagcatttctttccattgcgttttagagctagaaatagcaagttaaaataaggctagtccgttatcaacttgaaaaagtggcaccgagtcggtgctttt

SEQ ID NO: 5 - RNA, 100 nt, synthetic construct
  misc_feature 1..100: note = pH1N1 WT N1 (1-3, 97-99: 2'-O-methyl/ phosphorothioate modification)
  source 1..100: mol_type = other RNA
cctcttagtgataattaggggttttagagctagaaatagcaagttaaaataaggctagtccgttatcaacttgaaaaagtggcaccgagtcggtgctttt

SEQ ID NO: 6 - RNA, 100 nt, synthetic construct
  misc_feature 1..100: note = pH1N1/H275Y N1 (1-3, 97-99: 2'-O-methyl/ phosphorothioate modification)
  source 1..100: mol_type = other RNA
cctcttagtaataattaggggttttagagctagaaatagcaagttaaaataaggctagtccgttatcaacttgaaaaagtggcaccgagtcggtgctttt

SEQ ID NO: 7 - RNA, 100 nt, synthetic construct
  misc_feature 1..100: note = IFV H3 (1-3, 97-99: 2'-O-methyl/ phosphorothioate modification)
  source 1..100: mol_type = other RNA
cttccatttggagtgatgcagttttagagctagaaatagcaagttaaaataaggctagtccgttatcaacttgaaaaagtggcaccgagtcggtgctttt

SEQ ID NO: 8 - RNA, 100 nt, synthetic construct
  misc_feature 1..100: note = IFV H5 (1-3, 97-99: 2'-O-methyl/phosphorothioate modification)
  source 1..100: mol_type = other RNA
caaccatctaccattccctggttttagagctagaaatagcaagttaaaataaggctagtccgttatcaacttgaaaaagtggcaccgagtcggtgctttt

SEQ ID NO: 9 - RNA, 40 nt, synthetic construct
  misc_feature 1..40: note = SARS-CoV-2 N1
  source 1..40: mol_type = other RNA
gaccccaaaatcagcgaaatgcaccccgcattacgtttgg

SEQ ID NO: 10 - RNA, 38 nt, synthetic construct
  misc_feature 1..38: note = SARS-CoV-2 N2
  source 1..38: mol_type = other RNA
ttacaaacattggccgcaaattgcacaatttgccccca

SEQ ID NO: 11 - RNA, 38 nt, synthetic construct
  misc_feature 1..38: note = SARS-CoV-2 N3
  source 1..38: mol_type = other RNA
gggagccttgaatacaccaaaagatcacattggcaccc

SEQ ID NO: 12 - RNA, 44 nt, synthetic construct
  misc_feature 1..44: note = pH1N1 H1
  source 1..44: mol_type = other RNA
ggtaccgagatatgcattcgcaatggaaagaaatgctggatctg

SEQ ID NO: 13 - RNA, 48 nt, synthetic construct
  misc_feature 1..48: note = pH1N1 WT N1
  source 1..48: mol_type = other RNA
atcagtcgaaatgaatgcccctaattatcactatgaggaatgctcctg

SEQ ID NO: 14 - RNA, 48 nt, synthetic construct
  misc_feature 1..48: note = pH1N1/H275Y N1
  source 1..48: mol_type = other RNA
atcagtcgaaatgaatgcccctaattattactatgaggaatgctcctg

SEQ ID NO: 15 - RNA, 44 nt, synthetic construct
  misc_feature 1..44: note = IFV H3
  source 1..44: mol_type = other RNA
ttggcaagtgcaagtctgaatgcatcactccaaatggaagcatt

SEQ ID NO: 16 - RNA, 46 nt, synthetic construct
  misc_feature 1..46: note = IFV H5
  source 1..46: mol_type = other RNA
ggttttatagagggaggatggcagggaatggtagatggttggtatg

SEQ ID NO: 17 - RNA, 40 nt, synthetic construct
  misc_feature 1..40: note = SARS
  source 1..40: mol_type = other RNA
aacatgcttaggataatggcctctcttgttcttgctcgca

SEQ ID NO: 18 - RNA, 26 nt, synthetic construct
  modified_base 4: mod_base = OTHER; note = thymine
  modified_base 8: mod_base = OTHER; note = thymine
  modified_base 14: mod_base = OTHER; note = thymine
  modified_base 17..20: mod_base = OTHER; note = thymine
  modified_base 25: mod_base = OTHER; note = thymine
  misc_feature 1..26: note = SARS-CoV-2 N1
  source 1..26: mol_type = other RNA
gggtgcatcgggctgattttggggtc

SEQ ID NO: 19 - RNA, 26 nt, synthetic construct
  modified_base 2: mod_base = OTHER; note = thymine
  modified_base 7..8: mod_base = OTHER; note = thymine
  modified_base 18: mod_base = OTHER; note = thymine
  modified_base 20..22: mod_base = OTHER; note = thymine
  modified_base 24: mod_base = OTHER; note = thymine
  misc_feature 1..26: note = SARS-CoV-2 N2
  source 1..26: mol_type = other RNA
gtgcaattcggggccaatgtttgtaa

SEQ ID NO: 20 - RNA, 26 nt, synthetic construct
  modified_base 3: mod_base = OTHER; note = thymine
  modified_base 5..8: mod_base = OTHER; note = thymine
  modified_base 13: mod_base = OTHER; note = thymine
  modified_base 15..16: mod_base = OTHER; note = thymine
  modified_base 23: mod_base = OTHER; note = thymine
  misc_feature 1..26: note = SARS-CoV-2 N3
  source 1..26: mol_type = other RNA
gatcttttcgggtattcaaggctccc

SEQ ID NO: 21 - RNA, 32 nt, synthetic construct
  source 1..32: mol_type = other RNA
  misc_feature 1..32: note = chimeric
  modified_base 5: mod_base = OTHER; note = thymine
  modified_base 32: mod_base = OTHER; note = thymine
tccattgccggtgcatatctcggtaccaactt

SEQ ID NO: 22 - RNA, 26 nt, synthetic construct
  source 1..26: mol_type = other RNA
  misc_feature 1..26: note = chimeric
  modified_base 18: mod_base = OTHER; note = thymine
  modified_base 26: mod_base = OTHER; note = thymine
aattagggcggttcatttcgactgat

SEQ ID NO: 23 - RNA, 27 nt, synthetic construct
  source 1..27: mol_type = other RNA
  misc_feature 1..27: note = chimeric
  modified_base 5: mod_base = OTHER; note = thymine
  modified_base 16: mod_base = OTHER; note = thymine
  modified_base 23: mod_base = OTHER; note = thymine
gtgatgcacggagacttgcacttgcca

SEQ ID NO: 24 - RNA, 27 nt, synthetic construct
  source 1..27: mol_type = other RNA
  misc_feature 1..27: note = chimeric
  modified_base 3: mod_base = OTHER; note = thymine
  modified_base 7: mod_base = OTHER; note = thymine
  modified_base 15: mod_base = OTHER; note = thymine
  modified_base 21: mod_base = OTHER; note = thymine
  modified_base 23: mod_base = OTHER; note = thymine
attccctgcggtcctccctctataaaa

(19) United States
(12) Patent Application Publication
(10) Pub. No.: US 2023/0235382 A1
(43) Pub. Date: Jul. 27, 2023

(54) TARGET RNA DETECTION METHOD BASED ON DCAS9/GRNA COMPLEX

(71) Applicants: KOREA RESEARCH INSTITUTE OF BIOSCIENCE AND BIOTECHNOLOGY, Daejeon (KR); BIONANO HEALTH GUARD RESEARCH CENTER, Daejeon (KR)

(72) Inventors: Tae Joon KANG, Daejeon (KR); Jeong Moon, Daejeon (KR); Ju Yeon Jung, Daejeon (KR); Kyu Sun Lee, Daejeon (KR); Eun Kyung Lim, Daejeon (KR); Hong Ki Kim, Daejeon (KR); Hyun Ju Kang, Daejeon (KR)

(73) Assignees: KOREA RESEARCH INSTITUTE OF BIOSCIENCE AND BIOTECHNOLOGY, Daejeon (KR); BIONANO HEALTH GUARD RESEARCH CENTER, Daejeon (KR)

(21) Appl. No.: 18/163,936

(22) Filed: Feb. 3, 2023

Related U.S. Application Data
(63) Continuation of application No. PCT/KR21/10284, filed on Aug. 4, 2021.

(30) Foreign Application Priority Data
Aug. 4, 2020 (KR) 10-2020-0097531

(51) Int. Cl. C12Q 1/6834; C12Q 1/70

(52) U.S. Cl. CPC C12Q 1/6834; C12Q 1/70; C12Q 2600/112

(57) ABSTRACT

The present invention provides a target RNA detection method based on a dCas9/gRNA complex. A target RNA detection method according to the present invention can detect target RNA with the naked eye and without separate gene isolation and amplification steps, and, in particular, can rapidly and accurately detect target RNA through excellent target specificity and rapidity, and thus can exhibit excellent effects on the detection of various pathogens and/or viruses.

### CROSS-REFERENCE TO RELATED APPLICATIONS

This application is a Bypass Continuation of International Application No. PCT/KR2021/010284, filed on Aug. 4, 2021, which is based on and claims priority based on Korean Patent Application No. 10-2020-0097531, filed on Aug. 4, 2020, the entire disclosures are incorporated herein by reference.

### SEQUENCE LISTING

The content of the electronically submitted sequence listing, file name: Q284148 sequence listing as filed.XML; size: 46,873 bytes; and date of creation: Jan. 31, 2023, is hereby incorporated by reference in their entirety.

### TECHNICAL FIELD

The present disclosure relates to a target RNA detection method based on a dCas9/gRNA complex.

### BACKGROUND ART

A method of labeling and detecting nucleic acids that are difficult to be detected in natural state thereof has been applied to various fields of molecular biology or cell biology. Nucleic acids with labeled substances attached have been widely used in order to detect signals on southern blotting, northern blotting, in situ hybridization, and nucleic acid microarrays using specific hybridization reactions. A method of amplifying DNA and simultaneously labeling DNA using labeled monomers (labeled dNTPs) or labeled primers in a polymerase chain reaction (PCR) is known. The thus labeled DNA is able to be detected with a microarray.

The method of labeling nucleic acids simultaneously performing PCR has an advantage of not requiring a separate step for labeling, but has a disadvantage in that when a monomer labeled with a fluorescent dye or the like is used, PCR efficiency is lower than using an unlabeled monomer. In addition, since RNA is not able to be amplified by PCR, detecting RNA by PCR labeling requires a step of preparing cDNA through reverse transcription, and in particular, short RNAs such as microRNAs (miRNAs) have a problem in that cDNA preparation is cumbersome. Accordingly, there is an urgent need to develop a nucleic acid detection technology having more improved sensitivity and specificity.

The methods described above are easy to detect a nucleic acid to be targeted when a large amount of detection nucleic acid is present, and have been widely used today. Nevertheless, when a small amount of target nucleic acid is present, it is very difficult to detect the nucleic acid (low sensitivity), there are frequent cases that due to other inhibitors, it is impossible to detect a specific target only, but a non-specific target is detected by mistake (low specificity).

In addition, in order to cope with diseases caused by infections such as pathogens or viruses at an early stage and to prevent the progression and spread of diseases, it is necessary to diagnose quickly and accurately whether or not infection by pathogens or viruses has occurred. If the infection is able to be diagnosed during the incubation period which is the time between infection and the onset of symptoms or signs of infection, it is possible to effectively prevent the spread of infectious diseases and to block great damage. In other words, in order to cope with the spread of disease caused by virus infection at an early stage and to proceed with appropriate treatment for drug-resistant virus infection caused by single nucleotide sequence modification, it is required to quickly and accurately diagnose whether or not infection with the corresponding virus has occurred.

The CRISPR/Cas system is the immune system of bacteria, and plays a role in preventing infection from the outside by recognizing and cutting DNA/RNA introduced from the outside. In particular, since it was discovered that the CRISPR/Cas system is capable of performing sequence-specific recognition and cutting, this system has been attracting attention as a new gene editing technology, while simultaneously being applied in various ways, even including technologies for detecting and diagnosing target genes. However, there is still no technology for detecting target genes with the naked eye using the CRISPR/Cas system, and no research has been conducted to isolate viral genes and directly apply the genes to the CRISPR/Cas-based gene detection system without an amplification process.

Therefore, unlike conventional gene diagnosis methods in which PCR is necessarily accompanied or only genes are isolated and analyzed, there is a need to develop a CRISPR/Cas-based technology capable of detecting target genes with high sensitivity without performing a separate gene isolation step and PCR process.

### RELATED ART DOCUMENT

### Patent Document

Korean Patent Laid-Open Publication No. 10-2013-0094498

### DISCLOSURE

### Technical Problem

Under the above circumstances, the present inventors have made great efforts to develop a rapid and accurate gene detection method. As a result, the present inventors found that in detecting target RNA, when a dCas9/gRNA complex comprising inactivated Cas9 (dCas9) and a guide RNA that specifically binds to the target RNA, and a PAMmer are used, it was possible to simplify the complicated procedures of existing molecular diagnostic methods that necessarily include the gene amplification process while simultaneously overcoming disadvantages of immunodiagnosis with low sensitivity, and completed the target RNA specific detection method of the present disclosure.

Accordingly, an object of the present disclosure is to provide a target RNA detection method based on a PAMmer-introduced dCas9/gRNA system.

In addition, another object of the present disclosure is to provide a target RNA detection kit based on a PAMmer-introduced dCas9/gRNA system.

The following detailed description of the invention and claims will make other objects and advantages of the present disclosure more apparent.

### Technical Solution

Terms used herein are merely used for illustration purposes, which should not be construed as limiting the present disclosure. Singular expressions include plural expressions unless the context clearly indicates otherwise. In the present specification, terms such as “comprise” or “have” are intended to designate the presence of features, numbers, steps, operations, components, parts, or combinations thereof described in the specification and it should not be understood as precluding the possibility of the presence or addition of one or more other features, numbers, steps, operations, components, parts, or combinations thereof.

Further, unless defined otherwise, all terms used herein, including technical or scientific terms, have the same meaning as commonly understood by one of ordinary skill in the art to which the embodiments belong. Terms such as those defined in commonly used dictionaries should be interpreted as having a meaning consistent with the meaning in the context of the related art, and unless explicitly defined in this application, it is not to be construed in an idealized or overly formal sense.

As used herein, the terms “nucleic acid sequence,” “nucleotide sequence,” and “polynucleotide sequence” refer to an oligonucleotide or polynucleotide, to fragments or parts thereof, to DNA or RNA of genomic or synthetic origin which may be single-stranded or double-stranded, and to either the sense or antisense strand.

Hereinafter, the present disclosure will be described in more detail.

According to one aspect of the present disclosure, the present disclosure provides a target RNA detection method comprising:

(a) reacting a dCas9/gRNA complex with a PAMmer and a biological sample isolated from the subject, wherein the dCas9/gRNA complex includes inactivated Cas9 (dCas9) and a gRNA (guide RNA) complementary to a target RNA; and

wherein the PAMmer is an oligonucleotide in which a labeled ligand indirectly generating a detectable signal is bound to 3′-end, including a 3′-first hybridization region having a hybridization nucleotide sequence complementary to the target RNA, a protospacer-adjacent motif (PAM) sequence, and a 5′-second hybridization region having a hybridization nucleotide sequence complementary to the target RNA,

(b) treating a reaction product of step (a) with an anti-ligand that recognizes the detectable signal.

The target RNA detection method of the present disclosure comprises, in the presence of a PAMmer, the reacting of a dCas9/gRNA complex and a sample containing a target RNA, wherein the dCas9/gRNA complex includes inactivated Cas9 (dCas9) and a guide RNA that specifically binds to the target RNA.

More specifically, the PAMmer is divided into the following three regions:

(i) a 3′-first hybridization region having a hybridization nucleotide sequence complementary to the target RNA, (ii) a protospacer-adjacent motif (PAM) sequence, and (iii) a 5′-second hybridization region having a hybridization nucleotide sequence complementary to the target RNA.

The 3′-first hybridization region is a region that specifically binds to the target RNA and that includes a labeled ligand bound to 3′-end, the labeled ligand indirectly generating a detectable signal; and

the 5′-second hybridization region specifically binds to the target RNA, and the sequence of the 5′-second hybridization region hybridized to the target RNA has the same sequence as the sequence of the gRNA present at a corresponding position thereof.

The step is to react a sample containing one or more genes containing a target gene with the PAMmer and the dCas9/gRNA complex, and through this reaction, it is possible to provide a reaction product containing a binding product in which the target gene, PAMmer, and the dCas9/gRNA complex are bound, genes other than the target gene that is not reacted, and the unreacted complex.

In the present disclosure, the complex including the inactivated Cas9 (dCas9) and the gRNA (guide RNA) specifically bound to the target RNA may be formed before performing the target RNA detection method, but is not limited thereto, and when performing the steps, dCas9 and guide RNA may be formed either sequentially or together by reacting with the sample.

As used herein, the term “biological sample” means any sample containing any RNA and/or target RNA. The biological sample may be any tissue or body fluid obtained from a subject.

The biological sample includes, but not limited to, a subject's sputum, blood, serum, plasma, blood cells (for example, white blood cells), tissues, biopsy samples, smear samples, washing samples, swab samples, body fluids containing cells, mobile nucleic acid, urine, peritoneal fluid and pleural fluid, cerebrospinal fluid, feces, lacrimal fluid or cells therefrom. Biological samples may also include tissue sections taken for histological purposes, i.e., frozen or immobilized sections or microdissected cellular or extracellular portions thereof. The biological sample may be obtained by a method that does not adversely affect the subject.

In the present disclosure, the term “guide RNA” is a piece of RNA comprising a sequence that specifically binds to a target RNA, and the guide RNA of the present disclosure may form a complex with the Cas9 protein. The guide RNA may be composed of crRNA (CRISPR RNA) and tracrRNA (trans-activating crRNA).

The crRNA may bind to the target RNA.

The tracrRNA may act to change the structure of the dCas9 protein by binding to crRNA.

Specifically, the guide RNA used herein may be sgRNA (single chain guide RNA) linked in one strand while maintaining the roles of crRNA and tracrRNA.

It is preferable that the guide RNA has a complementary sequence at the 3′-end based on the sequence of the target RNA, and the PAMmer has a complementary sequence at the 5′-end based on the sequence of the target RNA.

The guide RNA (gRNA) may comprise the same sequences as the PAMmer (a nucleotide sequence in which a labeled ligand capable of indirectly generating a detectable signal is bound to 3′-end, including a PAM sequence and sequences complementary to a target RNA) that each complementarily bind to the target RNA, by 5 to 20 nucleotides in length, more preferably 6 to 10 nucleotides in length.

According to an embodiment according to the present disclosure, the biotin-PAMmer is composed of nucleotide sequences complementary to the target gene, but may include a PAM (5′-NGG-3′) mismatch site. The length was extended by 8 base pairs (bp) in the 3′ to 5′ direction from the PAM site, and this extended region was configured to overlap the target gene binding region of the gRNA. At the same time, biotin was bound to 3′ end.

As used herein, the term “specific binding” may be used interchangeably with hybridization.

The specific binding of the guide RNA to the target RNA may indicate that the guide RNA having sequences complementary to the target RNA hybridizes with the single-stranded target sequence of the target gene to form a double-stranded molecule (hybrid).

A sequence of the guide RNA complementary to the target RNA may hybridize with a portion of the target RNA, and the complementary sequence may be a sequence complementary to a portion of the target RNA by at least 90%, specifically at least 95%, and more specifically at 100%.

As used herein, the term “Cas protein” is a major protein component of the CRISPR/Cas system, and forms a complex with crRNA (CRISPR RNA) and tracrRNA (trans-activating crRNA) to form an activated endonuclease or nickase. The Cas protein may be a Cas9 protein, but is not limited thereto. In addition, the Cas9 protein may be derived from Streptococcus pyogens, but is not limited thereto.

In the present disclosure, the term “inactivated Cas9” refers to a Cas9 nuclease protein in which the nuclease function is inactivated, and may also be referred to as dCas9 (catalytically deficient Cas9). The production of inactivated Cas9 protein may be performed according to a conventional method for inactivating nuclease activity, but is not limited thereto. Information on dCas9 can be found from known documents, such as “A Programmable Dual-RNA-Guided DNA Endonuclease in Adaptive Bacterial Immunity (Martin Jinek et al, Science 17 Aug 2012: Vol. 337, Issue 6096, pp. 816-821)”. The above documents are incorporated herein by reference.

The Cas9 protein and genetic information thereof may be obtained from known databases such as GenBank of National Center for Biotechnology Information (NCBI).

The protospacer-adjacent motif (PAM) sequence is a short (2 to 6) nucleotide sequence that is essential for the Cas9 protein to precisely bind to and cut out the nucleotide sequence of the target RNA. PAM is able to function smoothly only when the PAM is present next to the target RNA. PAM is required to have the form ‘NGG’ in which two consecutive guanines (GG) are linked. In other words, the NGG necessarily includes GG, such as TGG, AGG, GGG, and CGG, and thus NGG or NGGNG, where N may be defined as any nucleotide.

Due to the presence of PAM, the Cas9 protein is expressed only at a specific region, and the Cas9 protein cuts the space between the 3rd base pair and the 4th base pair of the PAM sequence.

The PAM sequence may be located downstream of an overlapping sequence for the guide RNA and the target RNA based on the 5′-end of the nucleotide sequence including the PAM. In other words, the above PAM sequence may be located downstream about 5 to 12 bp, more preferably 6 to 10 bp from the 5′-end.

There is provided a nucleotide sequence in which a sequence complementary to the target RNA is present again downstream the PAM position sequence of the nucleotide sequence including the PAM, and a labeled ligand capable of indirectly generating a detectable signal is bound to 3′-end. In other words, in the nucleotide sequence including PAM, the PAM sequence is present between the sequences complementary to the target RNA.

The term “detectable signal” refers to a signal capable of being directly sensed by the human eye or by means of a detection system. The characteristics of the signal vary depending on the characteristics of the label used. In particular, the signal may be a colored, luminescent, fluorescent, phosphorescent, radioactive or magnetic signal. Preferably the signal is a colored signal.

As used herein, the term “indirect” when referring to the label means that the label is capable of generating a detectable signal only after interaction with another compound such as a substrate or binding partner. The label capable of generating a detectable signal indirectly may be, for example, a first member of a ligand/anti-ligand pair or an enzyme that produces a detectable signal in the presence of a substrate.

Examples of the ligand/anti-ligand pairs contemplated in methods according to the present disclosure include, but are not limited to, the following pairs: biotin/avidin or avidin analogs, antigens/antibodies, in particular biotin/anti-biotin antibodies or digoxigenin/anti-digoxigenin antibodies, molecules/receptors or sugars/lectins.

In addition, for example, the labeled ligand bound to 3′-end may be specifically at least any one selected from the group consisting of biotin, digoxigenin, aptamers, peptides, fluorescent compounds, oligonucleotides, and polysaccharides.

Preferably, there is provided a nucleotide sequence in which a sequence complementary to the target RNA is present downstream the PAM sequence, and biotin is bound to 3′-end.

In the case of the 3′-end nucleotide sequence, biotin may be bound to the end of the sequence complementary to the target RNA, or biotin may be linked after further including an additional nucleotide sequence of 1 to 10 bp at the end of the complementary sequence.

The nucleotide sequence in which biotin is bound to 3′-end as a labeled marker, including a protospacer-adjacent motif (PAM) sequence and sequences complementary to the target RNA provides the labeled marker at the cut and detectable 3′-end due to the dCas9/gRNA complex.

Cas9 may also cut ssDNA by providing a PAM-presenting oligonucleotide (PAMmer) that binds to ssDNA. In a similar way, PAMmers may also be used to engineer Cas9 to cut ssRNA. In order to cut only RNA without touching DNA, PAMmers should target the RNA portion of the DNA that does not contain the PAM. The RNA-targeting Cas9 as described above is called RCas9, and has the simplicity of only designing the PAMmer complementary to the target and the gRNA.

The term PAMmer refers to an oligonucleotide including a PAM sequence capable of interacting with a guide nucleotide sequence-programmable RNA binding protein. Details of suitable PAMmer sequences are described, for example, in the document [O'Connell et al., Nature, 2014, 516:263-266].

The PAMmer is a short oligonucleotide designed to contain a PAM sequence while simultaneously containing a labeled ligand to generate a detectable signal so that single-stranded target RNA that does not contain a PAM sequence is able to be recognized by the dCas9/gRNA complex.

The PAM sequence refers to a protospacer-adjacent motif comprising about 2 to about 10 nucleotides. The PAM sequences are specific to the guide nucleotide sequence-programmable RNA binding proteins to which they bind and which are known in the art. For example, the Streptococcus pyogenes PAM has the sequence 5′-NGG-3′, where “N” is any nucleobase accompanied by two guanine (“G”) nucleobases.

The target RNA detection method of the present disclosure comprises (b) treating a reaction product of step (a) with an anti-ligand that recognizes the detectable signal.

Specifically, the target RNA detection method of the present disclosure comprises (b) treating a reaction product of step (a) with avidin or an avidin analog.

More specifically, the target RNA detection method of the present disclosure comprises (b) treating a reaction product of step (a) with a horseradish hydrogen peroxide conjugate of avidin or an avidin analog, and with a horseradish hydrogen peroxide substrate.

In the present disclosure, the step of treating the reaction product of step (a) with an anti-ligand that recognizes the detectable signal is to treat an anti-ligand substance capable of recognizing the labeled ligand capable of indirectly generating the detectable signal at the 3′-end according to step (a) above.

The anti-ligand substance capable of recognizing a labeled ligand capable of indirectly generating the detectable signal at the 3′-end may be, for example, at least any one selected from the group consisting of avidin or avidin analogs, antibodies (for example, an anti-biotin antibody and an anti-digoxigenin antibody), receptors, and lectins.

Based on the above ligand/anti-ligand pair, color formation may be achieved through an enzyme that produces a detectable signal in the presence of a substrate and the substrate thereof.

For example, the enzyme is horseradish peroxidase, alkaline phosphatase or β-galactosidase. The anti-ligand substance may be provided in a conjugated form with the above enzyme. For example, horseradish hydrogen peroxide conjugates of avidin or avidin analogues, and the like, may be included.

Regarding the substrate for the enzyme, for example, substrates for horseradish peroxidase may include 3,3′,5,5′-tetramethylbenzidine (TMB), 2,2′-azino-di-[3-ethylbenzthiazoline-6-sulfonic acid] (ABTS), o-phenylenediamine dihydrochloride (OPD), 3,3′-diaminobenzidine (DAB), luminol, and the like, and substrates for alkaline phosphatase may include p-nitrophenyl phosphate, disodium salt (PNPP), and the like, and substrates for β-galactosidase may include chlorophenol red-B-D galactopyrano (CPRG), O-nitrophenyl-β-D-galactopyranoside (ONPG), 5-bromo-4-chloro-3-Indolyl-β-D-galactoside (X-Gal), and the like.

The generation of a detectable signal through the ligand/anti-ligand as described above provides information to enable specific detection of target RNA with high sensitivity even without performing the step of isolating the gene and/or RNA from the cell lysate.

Preferably, avidin or an avidin analog may be treated to provide an anti-ligand, and then the anti-ligand may be treated with an enzyme that generates a detectable signal and a substrate. For example, avidin or the avidin analog may be treated with horseradish hydrogen peroxide reactive thereto, and a substrate applicable thereto, such as 3,3′,5,5′-tetramethylbenzidine (TMB), 2,2′-azino-di-[3-ethylbenzthiazoline-6-sulfonic acid] (ABTS), o-phenylenediamine dihydrochloride (OPD), 3,3′-diaminobenzidine (DAB), luminol, or the like, or may be treated with alkaline phosphatase and p-nitrophenyl phosphate disodium salt (PNPP), or the like.

More preferably, in detecting the target RNA, the nucleotide sequence in which biotin is bound to 3′-end, including a protospacer-adjacent motif (PAM) sequence and sequences complementary to the target RNA may be treated with a horseradish hydrogen peroxide conjugate of avidin or an avidin analog, followed by treatment with TMB (3,3′,5,5′-tetramethylbenzidine), thereby providing color formation information and/or fluorescent color change information. Here, the avidin analog may be, for example, streptavidin, neutravidin, or captavidin.

Accordingly, the target RNA may be specifically detected with high sensitivity without performing the step of isolating the gene and/or RNA from the cell lysate.

Accordingly, the target RNA detection method may further comprise: (c) confirming the fluorescent color change of a reaction product obtained in step (b) with the naked eye.

The type of target for detection is not limited as long as the target is capable of being detected according to the method of the present disclosure, but the provision of information as described above may be preferably employed for viruses, pathogens, and the like.

For example, a virus requiring rapid diagnosis may be a DNA virus, an RNA virus, or a retrovirus. Particularly, the RNA virus is preferred. In other words, the target RNA may be virus-derived RNA.

Specifically, examples of RNA viruses include at least one (or any combination thereof) of Coronaviridae, Picornaviridae, Caliciviridae, Flaviviridae, Togaviridae, Bornaviridae, Filoviridae, Paramyxoviridae, Pneumoviridae, Rhabdoviridae, Arenaviridae, Bunyaviridae, Orthomyxoviridae, or Deltaviruses. In some exemplary embodiments, virus is coronavirus, SARS, poliovirus, rhinovirus, hepatitis A virus, norwalk virus, yellow fever virus, West Nile virus, hepatitis C virus, dengue fever virus, zika virus, rubella virus, Ross River virus, sindbis virus, chikungunya virus, borna disease virus, ebola virus, marburg virus, measles virus, mumps virus, Nipah virus, Hendra virus, Newcastle disease virus, human respiratory syncytial virus, rabies virus, Lassa virus, hantavirus, Crimean-Congo hemorrhagic fever virus, influenza, or hepatitis D virus.

More preferably, the virus may be severe acute respiratory syndrome coronavirus 2 (SARS-CoV2) (COVID-19) or an influenza virus.

In addition, the detection method according to the present disclosure exhibits excellent sensitivity and accuracy even for single nucleotide mutations, thereby being also excellent for detecting viral mutations. For example, the viral mutation may include, but not limited to, MERS virus I529T and/or D510G mutation, polio virus VP1-101 and/or VP1-102 mutation, human immunodeficiency virus (HIV) V106A, V179D, and/or Y181C mutation, Zika virus S139N mutation, severe acute respiratory syndrome (SARS) D614G mutation, influenza virus H275Y mutation, and the like.

In another general aspect, the present disclosure provides a target RNA detection kit comprising:

(a) a dCas9/gRNA complex immobilized on a substrate surface, wherein the dCas9/gRNA complex includes dCas9 and a guide RNA (gRNA) complementary to a target RNA;

(b) PAMmer in which biotin is bound to 3′-end, including a 3′-first hybridization region having a hybridization nucleotide sequence complementary to the target RNA, a protospacer-adjacent motif (PAM) sequence, and a 5′-second hybridization region having a hybridization nucleotide sequence complementary to the target RNA; and

(c) an anti-ligand that recognizes the detectable signal.

The target RNA detection kit according to the present disclosure may provide rapid and accurate diagnostic information in that it is possible to detect the target RNA with the naked eye and without separate gene isolation and amplification steps, and, in particular, it is also possible to detect the single mutation target RNA with excellent sensitivity and accuracy.

Thus, the target RNA detection kit according to the present disclosure comprises (a) a dCas9/gRNA complex immobilized on a substrate surface, wherein the dCas9/gRNA complex includes dCas9 and a guide RNA (gRNA) complementary to a target RNA.

In the present disclosure, the immobilization indicates that the dCas9/gRNA complex is coated on the surface by treating the dCas9/gRNA complex on the substrate surface, which is a solid support, followed by incubation, but is not limited thereto as long as it is possible to achieve the object of the present disclosure. The immobilization may be further performed using any immobilization method known in the art.

According to the present disclosure, the dCas9/gRNA complex is immobilized on the substrate surface. The immobilized dCas9/gRNA complex facilitates reaction with the nucleotide sequence in which the labeled ligand capable of indirectly generating a detectable signal is bound to 3′-end, including the protospacer-adjacent motif (PAM) sequence and the sequences complementary to the target RNA, and provides a subsequently detectable labeled ligand rapidly produced by the target RNA.

The target RNA detection kit according to the present disclosure has (b) a nucleotide sequence in which a labeled ligand indirectly generating a detectable signal is bound to 3′-end, including a protospacer-adjacent motif (PAM) sequence and sequences complementary to the target RNA.

When the above nucleotide sequence is treated with the dCas9/gRNA complex immobilized on the substrate surface, different reaction results may be shown depending on the presence or absence of the target RNA.

The target RNA detection kit according to the present disclosure has (c) an anti-ligand that recognizes the detectable signal.

The anti-ligand provides information such as a colored, luminescent, fluorescent, phosphorescent, radioactive or magnetic signal in response to the labeled ligand providing a detectable signal, and thus provides information on the presence or absence of the target RNA.

The present disclosure provides a target RNA detection kit comprising: (a) a dCas9/gRNA complex immobilized on a substrate surface, wherein the dCas9/gRNA complex includes dCas9 and a guide RNA (gRNA) complementary to a target RNA;

(b) a nucleotide sequence in which biotin is bound to 3′-end, including a protospacer-adjacent motif (PAM) sequence and sequences complementary to the target RNA;

(c) a horseradish hydrogen peroxide conjugate of avidin or an avidin analog; and

(d) a horseradish hydrogen peroxide substrate.

The information described in the detection method above may be applied by appropriate modification to the present kit.

Further, in the kit, an optimal amount of reagents to be used in a particular reaction may be easily determined by those skilled in the art having the knowledge of the disclosure herein. Typically, the kit of the present disclosure is manufactured in a separate package or compartment including the above described components.

In addition, the kit may further comprise instructions for use and other tools or equipment necessary for detection.

With the PAMmer-introduced dCas9/gRNA complex-based system according to the present disclosure, it is possible to detect the target gene with the naked eye with high sensitivity without performing a conventional PCR process. Therefore, the present disclosure is able to effectively detect a plurality of target sequences simultaneously with even improved accuracy and convenience, and to precisely detect a target sequence even in a single base unit.

### Advantageous Effects

A target RNA detection method by a PAMmer-introduced dCas9/gRNA complex-based detection system with the naked eye according to the present disclosure may detect a target RNA with the naked eye and without separate gene isolation and amplification steps, and, in particular, may rapidly and accurately detect target RNA through excellent target specificity and rapidity, and thus may exhibit excellent effects on the detection of various pathogens and/or viruses.

### DESCRIPTION OF DRAWINGS

FIG. 1a shows a nucleotide sequence structure of target RNA, biotin-PAMmer, and gRNA, and FIG. 1b shows electrophoresis results confirming a change in a mobile phase of the target RNA/biotin-PAMmer when the target RNA and biotin-PAMmer react with a dCas9/gRNA complex.

FIG. 2 shows a result of confirming the surface immobilization of the dCas9/gRNA complex.

(a) of FIG. 3 shows a nucleotide sequence structure of SARS-CoV-2 N1 (target RNA), biotin-PAMmer, and gRNA, (b) of FIG. 3 shows the results of quantitative detection of the SARS-CoV-2 N1 gene with the naked eye using the dCas9/gRNA complex and biotin-PAMmer, (c) of FIG. 3 shows a nucleotide sequence structure of pH1N1 H1 (target RNA), biotin-PAMmer, and gRNA, and (d) of FIG. 3 shows the result of quantitative detection of the pH1N1 H1 gene with the naked eye using the dCas9/gRNA complex and biotin-PAMmer.

(a) of FIG. 4 shows the results of selective detection of SARS-CoV-2 and pH1N1 H1 genes using dCas9/gRNA complex and biotin-PAMmer, and (b) of FIG. 4 shows the results of selective detection of influenza virus subtype (H1, H3, and H5) genes using the dCas9/gRNA complex and biotin-PAMmer.

FIG. 5 shows detection results of drug-resistant influenza virus genes based on the dCas9/gRNA complex, wherein (a) and (b) of FIG. 5 show sequence information, and (c) and (d) of FIG. 5 show detection results of drug-resistant influenza virus.

FIG. 6 shows results of selective detection of SARS-CoV-2 and pH1N1 genes without separate gene isolation and amplification steps from the virus culture medium, wherein (a) of FIG. 6 shows a related schematic diagram, and (b) of FIG. 6 shows experimental results.

FIG. 7 shows results obtained by treating SARS-CoV-2, pH1N, and drug-resistant pH1N1 in negative nasopharyngeal aspirate and sputum samples, respectively, and then detecting the treated virus without separate gene isolation and amplification, wherein (a) of FIG. 7 shows a schematic diagram and (b) to (d) of FIG. 7 show detection results.

FIG. 8 shows results of detecting COVID-19 in the nasopharyngeal aspirate and sputum samples of COVID-19 positive patients without separate gene isolation and amplification steps. (samples of 3 negative patients and 5 positive patients were used.)

FIG. 9 shows results of detecting COVID-19 in the nasopharyngeal aspirate and sputum samples of COVID-19 positive patients without separate gene isolation and amplification steps. (samples of 10 negative patients and 21 positive patients were used.)

### BEST MODE

Hereinafter, the present disclosure will be described in more detail through Examples. However, these Examples are provided to illustrate the present disclosure by way of example, and the scope of the present disclosure is not limited to these Examples.

### Example 1: Target Specificity of dCas9/gRNA Through Introduction of Biotin-PAMmer

In order to demonstrate the target-specific detection effect by the dCas9/gRNA system through introduction of the biotin-PAMmer of the present disclosure, the present inventors confirmed the target specificity of the dCas9/gRNA complex in the presence of target RNA and biotin-PAMmer.

A brief description is as follows: First, a dCas9/gRNA complex was formed by reacting 100 nM of gRNA and 1 μM of dCas9 protein at room temperature for 10 minutes.

In addition, the PAMmer is a short oligonucleotide designed to contain a PAM sequence while simultaneously containing a labeled ligand to generate a detectable signal so that single-stranded target RNA that does not contain a PAM sequence is able to be recognized by the dCas9/gRNA complex.

The PAMmer of the present disclosure is an oligonucleotide including a PAM sequence capable of interacting with a guide nucleotide sequence-programmable RNA binding protein,

which includes nucleotide sequence regions (3′-first hybridization region and 5′-second hybridization region) complementary to the target gene (RNA) and the PAM sequence; and

includes a labeled ligand indirectly generating a detectable signal at the 3′-end (3′-first hybridization region) of the oligonucleotide;

wherein the 5′-second hybridization region is a region extended by 8 base pairs in the 5′-end direction from the PAM sequence, and the extension site is designed to match (sequence is the same) with the target gene binding (hybridization) region of the gRNA.

In the present Example, biotin-PAMmer in a biotin-bound form was used.

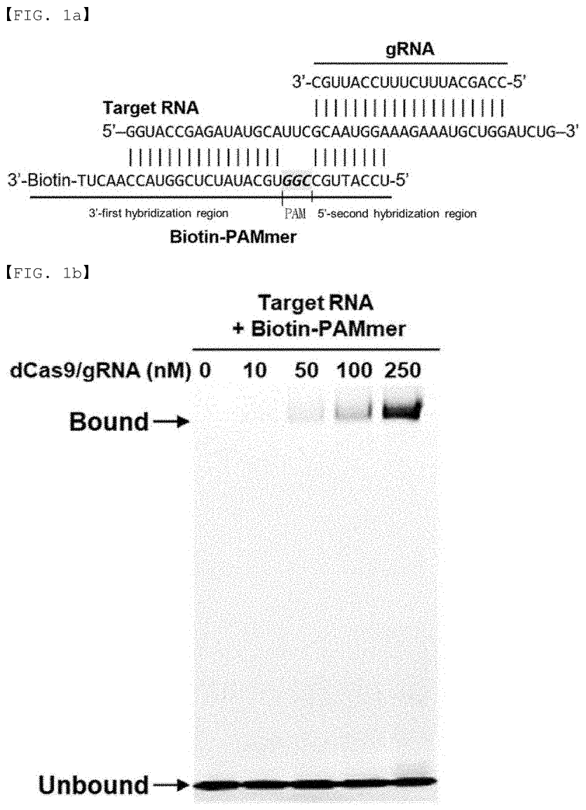

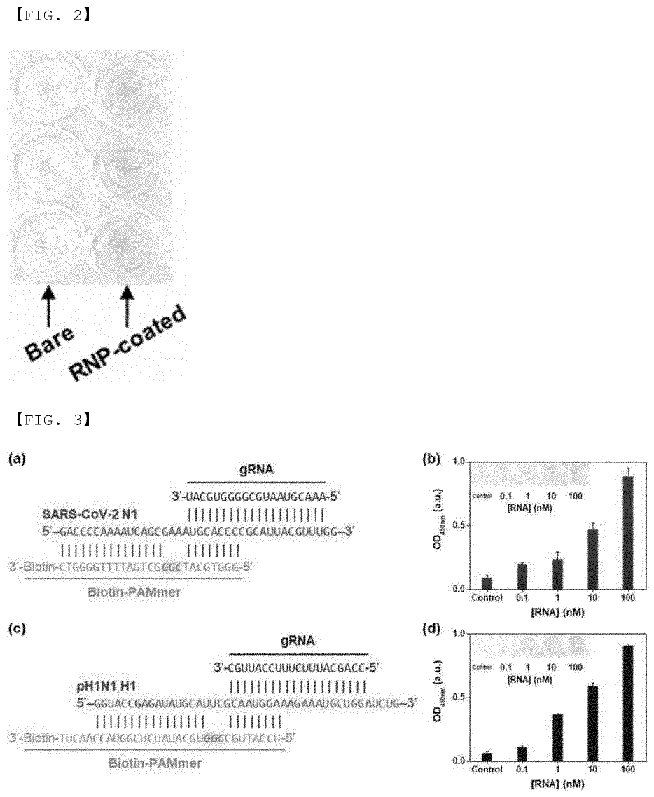

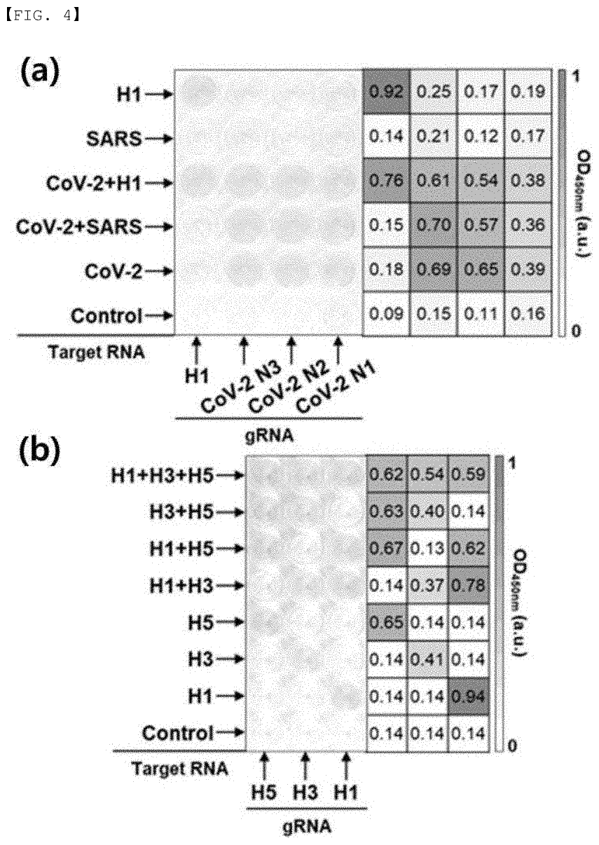

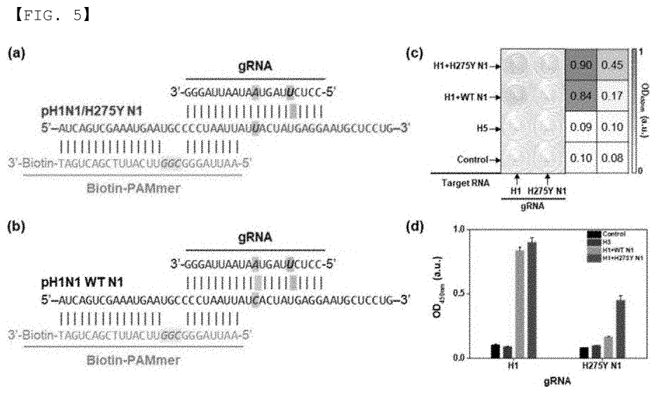

The dCas9/gRNA complex was diluted by concentration (10, 50, 100, and 250 nM), and 1 μM of the target RNA gene, 1 μM of biotin-PAMmer, and 1× reaction buffer were mixed therewith, followed by reaction at 37° C. for 1 hour. The reaction product was subjected to electrophoresis using 8% native PAGE gel, and then the mobility shift of biotin-PAMmer and target RNA was confirmed. The nucleotide sequence structure of gRNA, biotin-PAMmer, and target RNA used in the reaction is shown in FIG. 1a.

As a result, as shown in FIG. 1b, it was confirmed that in the presence of target RNA and biotin-PAMmer, when the dCas9/gRNA complex was not treated, there was no change in the mobile phase of biotin-PAMmer and target RNA, whereas when the dCas9/gRNA complex was treated, as the concentration of the complex increased, the mobile phase changed, specifically, amounts of biotin-PAMmer and target RNA increased.

It was confirmed from the above experiment that the dCas9/gRNA complex specifically bound to the biotin-PAMmer and the target RNA.

### Example 2: Immobilization of dCas9/gRNA Complex on Solid Phase

In order to confirm that the biotin-PAMmer-introduced dCas9/gRNA system according to the present disclosure could operate when the dCas9/gRNA complex was immobilized on a solid phase, the present inventors immobilized the dCas9/gRNA complex on the surface of a solid substrate.

A brief description is as follows: A dCas9/gRNA complex was formed by reacting 600 nM of gRNA and 1 μM of dCas9 at room temperature for 10 minutes and then the dCas9/gRNA complex diluted 10 times with 1× PBS solution was treated in a 96-well plate and reacted at room temperature for 2 hours.

Then, a surface was washed using a washing buffer containing 1× PBS and 0.05% tween 20. Next, the surface was treated with 0.1 mg/mL of bovine serum albumin (BSA) and reacted at room temperature for 40 minutes, and the surface was washed with a washing buffer. Then, the surface was treated with Cas9 monoclonal antibody diluted in 5% skim milk powder and reacted for 1 hour. After washing the surface using washing buffer, the surface was treated with HRP-conjugated anti-mouse IgG secondary antibody diluted in 5% skim milk and reacted for 1 hour. The surface was washed and sequentially treated with a TMB solution and a 2.5 M sulfuric acid solution to confirm a color change.

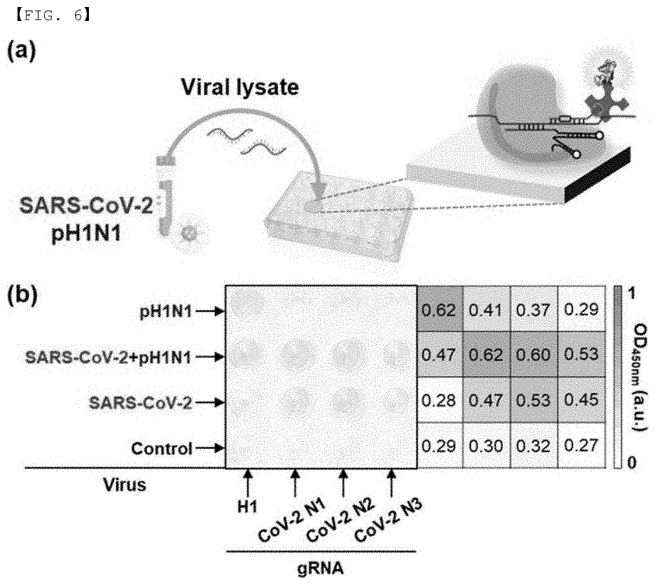

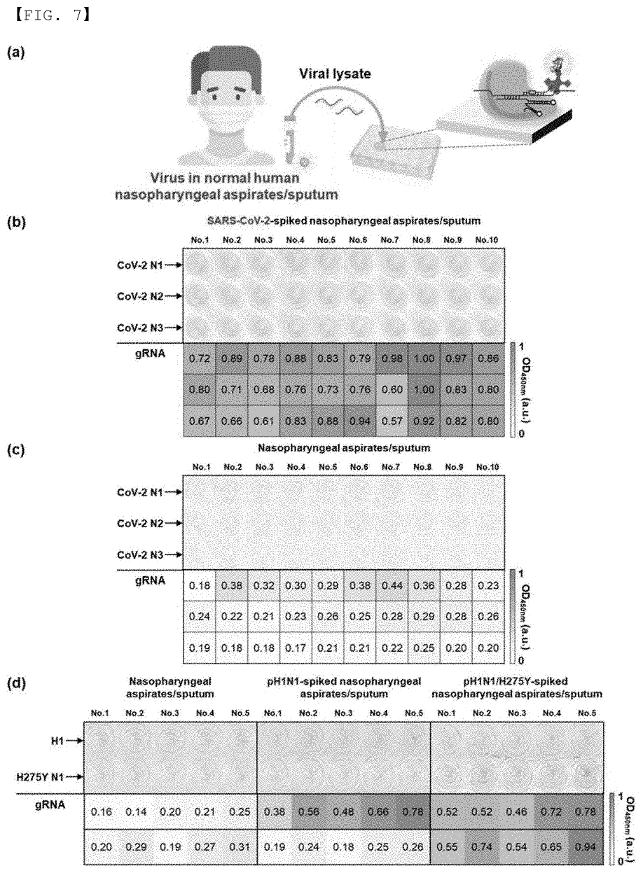

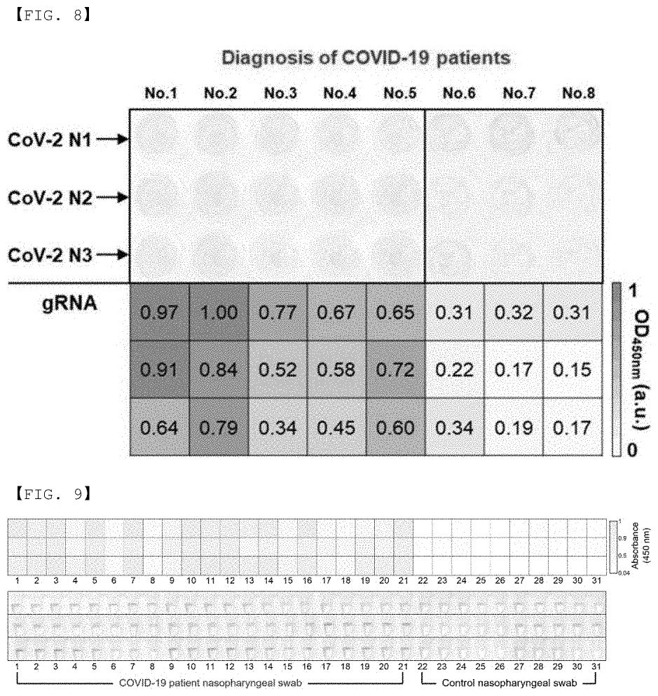

As a result, as shown in FIG. 2, it was confirmed that the color change occurred only when the dCas9/gRNA complex was treated compared to the surface where the dCas9/gRNA complex was not treated. From this confirmation, it could be appreciated that the dCas9/gRNA complex was successfully coated on the surface.

In other words, it is demonstrated that even if it is not performed under specific immobilization conditions and/or using a dedicated buffer for solid surface immobilization commonly known in the art, it is possible to perform the detection by application on the solid support only including treatment of the diluted dCas9/gRNA complex on the solid support such as the 96-well plate, followed by incubation at room temperature.

### Example 3: Detection of Target RNA with Naked Eye

The present inventors confirmed whether the target RNA could be detected with the naked eye by reacting the target RNA and biotin-PAMmer to the solid surface-immobilized dCas9/gRNA complex of Example 2, Followed by Treatment With Streptavidin-HRP and TMB.

Specifically, a dCas9/gRNA complex was formed by reacting 600 nM of gRNA and 1 μM of dCas9 at room temperature for 10 minutes and then the dCas9/gRNA complex diluted 10 times with 1× PBS solution was treated in a 96-well plate and reacted at room temperature for 2 hours. Then, a surface was washed using a washing buffer containing 1× PBS and 0.05% tween 20. Next, the surface was treated with 0.1 mg/mL of bovine serum albumin (BSA) and reacted at room temperature for 40 minutes, and the surface was washed with a washing buffer. Then, target RNA (0 to 100 nM) prepared for each concentration was mixed with 1 μM of biotin-PAMmer and 1× reaction buffer, and reacted on the surface at 37° C. for 1 hour. The surface was washed and reacted with 20 μg/mL of streptavidin-HRP for 30 minutes at room temperature. The surface was washed and sequentially treated with a TMB solution and a 2.5 M sulfuric acid solution to confirm a color change, and the absorbance was measured with a microplate machine. The absorbance was observed at 450 nm.

Here, the nucleotide sequence structures of gRNA, biotin-PAMmer, and target RNA used in the reaction are shown in FIGS. 3a and 3c.

As a result, as shown in (b) and (d) of FIG. 3, it was confirmed that the measured absorbance increased as the concentration of the target RNA increased (0 to 100 nM).

It is demonstrated that when the surface-immobilized dCas/gRNA complex, biotin-PAMmer, streptavidin-HRP, and TMB are reacted according to the present disclosure, it is possible to detect target RNA with the naked eye.

### Example 4: Specific Detection of Target RNA

The present inventors confirmed whether multiple genes could be simultaneously detected using dCas9/gRNA-based target RNA detection technology with the naked eye.

Specifically, as described in Example 3, dCas9/gRNA complexes targeting different genes were formed, immobilized on different surfaces of a 96 well plate, and treated with BSA. Then, samples in which several types of genes were mixed were simultaneously treated on the surfaces on which dCas9/gRNA complexes targeting different genes were immobilized. Then, as in Example 3, the detection reaction with the naked eye was performed through biotin-PAMmer and streptavidin-HRP treatment steps. Here, the biotin-PAMmer was designed to have a different nucleotide sequence for each target RNA, and each targeting gene was treated on the surface on which the corresponding dCas9/gRNA complex was immobilized.

Hereinafter, the sequence information used in the present Examples is shown in Table 1 below.

TABLE 1

| gRNA | Sequence (5′ to 3′) |
|---|---|
| SARS-Cov-2 N1 | mA*mA*mA* CGU AAU GCG GGG UGC AUG UUU UAG AGC UAG AAA |
| (SEQ ID NO: 1) | UAG CAA GUU AAA AUA AGG CUA GUC CGU UAU CAA CUU GAA |
| | AAA GUG GCA CCG AGU CGG UGC mU*mU*mU* U |
| SARS-Cov-2 N2 | mU*mG*mG* GGG CAA AUU GUG CAA UUG UUU UAG AGC UAG AAA |
| (SEQ ID NO: 2) | UAG CAA GUU AAA AUA AGG CUA GUC CGU UAU CAA CUU GAA |
| | AAA GUG GCA CCG AGU CGG UGC mU*mU*mU* U |
| SARS-Cov-2 N3 | mG*mG*mG* UGC CAA UGU GAU CUU UUG UAG AAA UAG CAA GUU |
| (SEQ ID NO: 3) | AAA AUA AGG CUA GUC CGU UAU CAA CUU GAA AAA GUG GCA |
| | CCG AGU CGG UGC mU*mU*mU* U |
| pH1N1 H1 | mC*mC*mA* GCA UUU CUU UCC AUU GCG UUU UAG AGC UAG AAA |
| (SEQ ID NO: 4) | UAG CAA GUU AAA AUA AGG CUA GUC CGU UAU CAA CUU GAA |
| | AAA GUG GCA CCG AGU CGG UGC mU*mU*mU* U |
| pH1N1 WT N1 | mC*mC*mU* CUU AGU GAU AAU UAG GGG UUU UAG AGC UAG AAA |
| (SEQ ID NO: 5) | UAG CAA GUU AAA AUA AGG CUA GUC CGU UAU CAA CUU GAA |
| | AAA GUG GCA CCG AGU CGG UGC mU*mU*mU* U |
| pH1N1/H275Y N1 | mC*mC*mU* CUU AGU AAU AAU UAG GGG UUU UAG AGC UAG AAA |
| (SEQ ID NO: 6) | UAG CAA GUU AAA AUA AGG CUA GUC CGU UAU CAA CUU GAA |
| | AAA GUG GCA CCG AGU CGG UGC mU*mU*mU* U |
| IFV H3 | mC*mU*mU* CCA UUU GGA GUG AUG CAG UUU UAG AGC UAG AAA |
| (SEQ ID NO: 7) | UAG CAA GUU AAA AUA AGG CUA GUC CGU UAU CAA CUU GAA |
| | AAA GUG GCA CCG AGU CGG UGC mU*mU*mU* U |
| IFV H5 | mC*mA*mA* CCA UCU ACC AUU CCC UGG UUU UAG AGC UAG AAA |
| (SEQ ID NO: 8) | UAG CAA GUU AAA AUA AGG CUA GUC CGU UAU CAA CUU GAA |
| | AAA GUG GCA CCG AGU CGG UGC mU*mU*mU* U |
| Target | Sequence (5′ to 3′) |
| SARS-Cov-2 N1 | GAC CCC AAA AUG AGC GAA AUG CAC CCC GCA UUA CGU UUG |
| (SEQ ID NO: 9) | G |
| SARS-Cov-2 N2 | UUA CAA ACA UUG GCC GCA AAU UGC ACA AUU UGC CCC CA |
| (SEQ ID NO: 10) | |
| SARS-Cov-2 N3 | GGG AGC CUU GAA UAC ACC AAA AGA UCA CAU UGG CAC CC |
| (SEQ ID NO: 11) | |
| pH1N1 H1 | GGU ACC GAG AUA UGC AUU CGC AAU GGA AAG AAA UGC UGG |
| (SEQ ID NO: 12) | AUG UG |
| pH1N1 WT N1 | AUG AGU CGA AAU GAA UGC CCC UAA UUA UCA CUA UGA GGA |
| (SEQ ID NO: 13) | AUG CUC CUG |
| pH1N1/H275Y N1 | AUG AGU CGA AAU GAA UGC CCC UAA UUA UUA CUA UGA GGA |
| (SEQ ID NO: 14) | AUG CUC CUG |
| IFV H3 | UUG GCA AGU GCA AGU CUG AAU GCA UCA CUC CAA AUG GAA |
| (SEQ ID NO: 15) | GCA UU |
| IFV H5 | GGU UUU AUA GAG GGA GGA UGG CAG GGA AUG GUA GAU GGU |
| (SEQ ID NO: 16) | UGG UAU G |
| SARS | AAC AUG CUU AGG AUA AUG GCC UCU CUU GUU CUU GCU CGC |
| (SEQ ID NO: 17) | A |
| Biotin-PAMmer | Sequence (5′ to 3′) |
| SARS-Cov-2 N1 | GGG TGC ATC GGG CTG ATT TTG GGG TC-Biotin |
| (SEQ ID NO: 18) | |
| SARS-Cov-2 N2 | GTG CAA TTC GGG GCC AAT GTT TGT AA-Biotin |
| (SEQ ID NO: 19) | |
| SARS-CoV-2 N3 | GAT CTT TTC GGG TAT TCA AGG CTC CC-Biotin |
| (SEQ ID NO: 20) | |
| pH1N1 H1 | rUrCrC rATrU GrCC rGGrU GrCA rUArU CrUC rGGrU ArCC |
| (SEQ ID NO: 21) | rArAC rUT-Biotin |
| pH1N1 WT N1 and | rAArU rUArG GrGC GrGrU rUCA rUrUT CGA CrUG AT-Biotin |
| PH1N1/H275Y N1 | |
| (SEQ ID NO: 22) | |
| IFV H3 | rGrUG rATrG CrArC GrGrA GrAC TrUrG rCAC rUTG rCrCA- |
| (SEQ ID NO: 23) | Biotin |
| IFV H5 | rArUT rCCrC TrGrC GrGrU CrCT CrCrC rUCT rATA rArAA- |
| (SEQ ID NO: 24) | Biotin |

m_*: 2′-O-methyl/phosphorothioate modification,
Seq No. 21~24: Chimeric (r_: chimeric)

As a result, as shown in FIG. 4, it could be confirmed that the target RNA could be detected very selectively by each dCas9/gRNA complex, and it was shown that highly specific detection with the naked eye could be performed depending on the nucleotide sequence of the target RNA.

Specifically, (a) of FIG. 4 shows the results for SARS-CoV-2 N1 (CoV-2 N1), SARS-CoV-2 N2 (CoV-2 N2), SARS-CoV-2 N3 (CoV-2 N3), and pH1N1 H1 (H1). As can be appreciated from the results, the target RNA could be clearly detected with the naked eye, confirming that the detection of the coronavirus could be achieved.

Further, (b) of FIG. 4 shows the results for pH1N1 H1 (H1), IFV H3 (H3), and IFV H5 (H5). As can be appreciated from the results, the target RNA could be clearly detected with the naked eye, confirming that the detection of the influenza virus could be achieved.

In other words, it was demonstrated that simultaneous detection of multiple target RNAs could be achieved through the dCas9/gRNA-based target RNA detection technology with the naked eye of the present disclosure.

### Example 5: Detection of Gene Having Difference in Single Nucleotide Sequence

Among drug-resistant viruses that are resistant to Oseltamivir, a treatment for swine flu (H1N1 influenza) virus, the H275Y N1 mutant type is known to show a difference in single nucleotide sequence as compared to the drug-susceptible wild type. For proper treatment, it is required to perform a rapid diagnosis of drug-resistant virus infection.

Therefore, in order to confirm whether the dCas9/gRNA-based target RNA detection technology with the naked eye of the present disclosure is able to distinguish a gene having a difference in single nucleotide sequence, the present inventors conducted experiments on the mutant pH1N1/H275Y N1 and the wild type pH1N1 WT N1.

Specifically, as shown in (a) and (b) of FIG. 5, a gRNA was constructed so that a region having a difference in nucleotide sequence (for example, a single nucleotide mutation) compared to the wild type virus on pH1N1/H275Y RNA, which is the target RNA, was selected as a gRNA binding site to which the gRNA binds, and one nucleotide sequence mismatch is present at a position spaced by 5 base pairs (bp) in the 5′-end direction from the position of the different nucleotide sequence on the gRNA.

Then, as described in Example 3, the detection with the naked eye was performed by forming dCas9/gRNA complex and immobilizing it on the solid phase surface, followed by treatment with biotin-PAMmer, streptavidin-HRP, and TMB.

As a result, as shown in (c) and (d) of FIG. 5, color change was observed when the gene of the H275Y N1 mutant type was treated on the surface immobilized with dCas9/gRNA composed of gRNA complementary to the H275Y mutant gene of influenza virus.

On the other hand, no color change was observed when the wild-type gene having a difference in single nucleotide sequence from the target RNA was treated.

This suggests that the dCas9/gRNA-based target RNA detection technology with the naked eye of the present disclosure is able to distinguish the gene having a difference in single nucleotide sequence.

### Example 6: Virus Detection in Virus Culture Medium

The present inventors attempted to selectively detect target viral RNA by using the dCas9/gRNA-based target RNA detection technology with the naked eye of the present disclosure, without gene extraction and amplification steps through a separate kit, in culture media in which SARS-CoV-2 and novel influenza virus were cultured ((a) of FIG. 6).

In more detail, SARS-CoV-2 at a concentration of 10³ PFU/mL, swine flu virus (pH1N1) at a concentration of 10⁴ PFU/mL, and a mixture of the two viruses (SARS-CoV-2 and swine flu) were prepared, and then the respective samples were treated with a TCEP/EDTA (final concentration 100 mM/1 mM) solution. Then, the reactants were sequentially heat-treated at 50° C. for 5 minutes and at 64° C. for 5 minutes and used as samples. Next, as described in Example 3 above, the detection with the naked eye was performed by immobilizing the dCas9/gRNA complexes targeting genes of SARS-CoV-2 and H1N1 influenza virus on the solid surface, followed by treatment with biotin-PAMmer, streptavidin-HRP, and TMB.

As a result, as shown in (b) of FIG. 6, no color change was observed in the condition where the virus was not treated, but when the SARS-CoV-2 virus solution was treated alone, color change was observed only on the surfaces immobilized with the dCas9/gRNA complexes complementary to the SARS-CoV-2 genes (CoV-2 N1, N2, and N3).

In addition, when the H1N1 influenza virus solution was treated alone, color change was observed only on the surface on which the dCas9/gRNA complex complementary to the swine flu gene (H1) was immobilized.

Further, it could be confirmed that in the condition of mixing the two viruses, color changes were observed on all surfaces on which the dCas9/gRNA complex complementary to SARS-CoV-2 and H1 was immobilized.

Through this observation, it could be confirmed that the virus gene in the virus culture medium was capable of being detected very selectively through the dCas9/gRNA-based target RNA detection technology with the naked eye and without separate gene isolation and amplification steps.

### Example 7: Confirmation of Target RNA Detection in Nasopharyngeal Aspirate and Sputum

Viruses that cause respiratory diseases, such as SARS-CoV-2 and H1N1 influenza virus, are generally extracted from nasopharyngeal aspirate or sputum by a viral RNA isolation kit and detected by RT-PCR.

Accordingly, the present inventors attempted to detect viral RNA from nasopharyngeal aspirates or sputum by using the dCas9/gRNA-based target RNA detection technology with the naked eye of the present disclosure and without the separate gene extraction step through the kit ((a) of FIG. 7).

Specifically, SARS-CoV-2 (10³ PFU/mL), swine flu (10⁴ PFU/mL), and H275Y drug-resistant swine flu (10⁴ PFU/mL) viruses were treated in nasopharyngeal aspirate or sputum. Then, the virus-treated nasopharyngeal aspirate and sputum were treated with a TCEP/EDTA (final concentration: 100 mM/1 mM) solution, sequentially heat-treated at 50° C. for 5 minutes and at 64° C. for 5 minutes, and then used as samples. As described above in Example 3, the detection with the naked eye was performed by immobilizing the dCas9/gRNA complex targeting genes of SARS-CoV-2, swine flu virus (pH1N1) and H275Y drug-resistant swine flu (pH1N1/H275Y) on the surface, followed by treatment with biotin-PAMmer, streptavidin-HRP, and TMB.

As a result, as shown in (b) to (c) of FIG. 7, no color change was observed in the negative nasopharyngeal aspirate and sputum sample conditions that were not treated with the virus, but it could be confirmed that the positive samples treated with the virus showed selective color change on the surface where each complementary dCas9/gRNA complex was immobilized.

Through this confirmation, it could be appreciated that target RNA detection could be performed through the dCas9/gRNA-based target RNA detection technology with the naked eye and without separate gene isolation and amplification steps from nasopharyngeal aspirate and sputum samples.

Specifically, it was confirmed in (b) and (c) of FIG. 7 that it was possible to confirm the presence or absence of SARS-Cov-2, and in (d) of FIG. 7 that it was also possible to identify not only the influenza virus but also variants having single mutations.

### Example 8: Confirmation of Detection of Target RNA in Nasopharyngeal Aspirate and Sputum of COVID-19 Positive Patients

In order to prove that COVID-19 was actually detectable in clinical practice by using the dCas9/gRNA-based target RNA detection technology with the naked eye and without the separate gene extraction step through a kit according to the related art, the present inventors confirmed the target RNA detection from the patient's nasopharyngeal aspirate and sputum.

Briefly, nasopharyngeal aspirate and sputum from COVID-19 positive and negative patients were treated with TCEP/EDTA (final concentration 100 mM/1 mM) solution, respectively, sequentially heat-treated at 50° C. for 5 minutes and at 64° C. for 5 minutes, and then used as samples. As described in Example 3 above, the detection with the naked eye was performed by immobilizing the dCas9/gRNA complex targeting a gene of SARS-CoV-2 on the surface, followed by treatment with biotin-PAMmer, streptavidin-HRP, and TMB.

As a result, as shown in FIG. 8, no color change was observed in the nasopharyngeal aspirate and sputum sample conditions of the negative patient, but the samples of the positive patient showed selective color change on the surface where each complementary dCas9/gRNA complex was immobilized.

The above experiment was conducted with 3 negative patients and 5 positive patients.

Accordingly, an additional experiment was conducted on more samples using the same experimental method. Specifically, samples of 21 positive patients and 10 negative patients were used in the additional experiment.

The result was shown in FIG. 9.

As shown in FIG. 9, no color change was observed in the samples of 10 negative patients, while a color change was observed in the samples of 21 positive patients, so that excellent efficacy of the detection method was confirmed.

Through this confirmation, it could be appreciated that COVID-19 infection could be diagnosed through the dCas9/gRNA-based target RNA detection technology with the naked eye and without separate gene isolation and amplification steps from nasopharyngeal aspirate and sputum samples.

From the above results, it was confirmed that the target RNA detection method according to the present disclosure could detect target RNA with the naked eye and without separate gene isolation and amplification steps, and in particular, could quickly and accurately detect the target RNA with excellent target specificity and rapidity. Therefore, it was demonstrated that the target RNA detection method could exhibit excellent effects in detecting various pathogens and/or viruses, in particular, highly prevalent viruses.

From the above description, those skilled in the art to which the present disclosure pertains will understand that the present disclosure may be embodied in other specific forms without changing the technical spirit or essential characteristics thereof. In this regard, it should be understood that the embodiments described above are illustrative in all respects and not restrictive. As the scope of the present disclosure, it should be construed that all changes or modifications derived from the meaning and scope of the claims to be described below and equivalents thereof rather than the above detailed description are included in the scope of the present disclosure.

1. A target RNA detection method comprising:
(a) reacting a dCas9/gRNA complex with a PAMmer and a biological sample isolated from the subject, wherein the dCas9/gRNA complex includes inactivated Cas9 (dCas9) and a gRNA (guide RNA) complementary to a target RNA; and
wherein the PAMmer is an oligonucleotide in which a labeled ligand indirectly generating a detectable signal is bound to 3′-end, including a 3′-first hybridization region having a hybridization nucleotide sequence complementary to the target RNA, a protospacer-adjacent motif (PAM) sequence, and a 5′-second hybridization region having a hybridization nucleotide sequence complementary to the target RNA,
(b) treating a reaction product of step (a) with an anti-ligand that recognizes the detectable signal.

2. The target RNA detection method of claim 1, wherein the labeled ligand capable of indirectly generating a detectable signal at the 3′-end in step (a) is at least one selected from the group consisting of biotin, digoxigenin, aptamers, peptides, fluorescent compounds, oligonucleotides, and polysaccharides.

3. The target RNA detection method of claim 1, wherein the anti-ligand that recognizes a detectable signal in step (b) is at least one selected from the group consisting of avidin or avidin analogs, antibodies, receptors, and lectins.

4. The target RNA detection method of claim 1, wherein the labeled ligand indirectly generating a detectable signal at the 3′-end in step (a) is biotin; and
the anti-ligand that recognizes a detectable signal in step (b) is avidin or an avidin analog.

5. The target RNA detection method of claim 1, wherein the gRNA in step (a) is a single chain guide RNA.

6. The target RNA detection method of claim 1, wherein the gRNA in step (a) contains the same sequence as the 5′-second hybridization region of the PAMmer, and the sequence is 5 to 20 nucleotides in length.

7. The target RNA detection method of claim 1, wherein the 5′-second hybridization region of the PAMmer in step (a) is 5 to 20 nucleotides in length in a 3′ to 5′ direction based on the PAM sequence.

8. The target RNA detection method of claim 1, wherein the PAM sequence in step (a) is 5′-NGG or NGGNG, where N is any nucleotide.

9. The target RNA detection method of claim 1, wherein the dCas9/gRNA complex in step (a) is immobilized.

10. The target RNA detection method of claim 4, wherein the avidin analog in step (b) is streptavidin, neutravidin, or captavidin.

11. The target RNA detection method of claim 4, wherein the avidin or avidin analog in step (b) is a horseradish hydrogen peroxide conjugate of avidin or the avidin analog.

12. The target RNA detection method of claim 11, wherein the horseradish hydrogen peroxide substrate in step (b) is any one selected from the group consisting of 3,3′,5,5′-tetramethylbenzidine (TMB), 2,2′-azino-di-[3-ethylbenzthiazoline-6-sulfonic acid] (ABTS), o-phenylenediamine dihydrochloride (OPD), 3,3′-diaminobenzidine (DAB), and luminol.

13. The target RNA detection method of claim 4, further comprising:
(c) confirming a color change of a reaction product obtained in step (b) with the naked eye.

14. The target RNA detection method of claim 1, wherein the target RNA is virus-derived RNA.

15. A target RNA detection kit comprising:
(a) a dCas9/gRNA complex immobilized on a substrate surface, wherein the dCas9/gRNA complex includes dCas9 and a guide RNA (gRNA) complementary to a target RNA;
(b) PAMmer in which a labeled ligand indirectly generating a detectable signal is bound to 3′-end, including a 3′-first hybridization region having a hybridization nucleotide sequence complementary to the target RNA, a protospacer-adjacent motif (PAM) sequence, and a 5′-second hybridization region having a hybridization nucleotide sequence complementary to the target RNA; and
(c) an anti-ligand that recognizes the detectable signal.

16. The target RNA detection kit of claim 15, wherein the labeled ligand indirectly generating a detectable signal at the 3′-end is any one selected from the group consisting of biotin, digoxigenin, aptamers, peptides, fluorescent compounds, oligonucleotides, and polysaccharides.

17. The target RNA detection kit of claim 15, wherein the anti-ligand that recognizes a detectable signal is any one selected from the group consisting of avidin or avidin analogs, antibodies, receptors, and lectins.

18. A target RNA detection kit comprising:
(a) a dCas9/gRNA complex immobilized on a substrate surface, wherein the dCas9/gRNA complex includes dCas9 and a guide RNA (gRNA) complementary to a target RNA;
(b) PAMmer in which biotin is bound to 3′-end, including a 3′-first hybridization region having a hybridization nucleotide sequence complementary to the target RNA, a protospacer-adjacent motif (PAM) sequence, and a 5′-second hybridization region having a hybridization nucleotide sequence complementary to the target RNA;
(c) a horseradish hydrogen peroxide conjugate of avidin or an avidin analog; and
(d) a horseradish hydrogen peroxide substrate.
